(19) United States
(12) Patent Application Publication
(10) Pub. No.: US 2021/0401401 A1
(43) Pub. Date: Dec. 30, 2021

(54) METHOD AND APPARATUS FOR CONTROLLING AN EYE LID DURING ULTRASOUND IMAGING

(71) Applicant: ArcScan, Inc., Golden, CO (US)

(72) Inventors: Erik Johan Giphart, Aurora, CO (US); Andrew K. Levien, Morrison, CO (US); Tom Wilmering, Eldorado Springs, CO (US); Barry Schafer, Folsum, CA (US)

(21) Appl. No.: 17/359,128

(22) Filed: Jun. 25, 2021

Related U.S. Application Data
(60) Provisional application No. 63/044,857, filed on Jun. 26, 2020.

(51) Int. Cl. A61B 8/00; A61B 8/10

(52) U.S. Cl. CPC A61B 8/40; A61B 8/10

(57) ABSTRACT

The present disclosure is directed to a method and apparatus for holding an eyelid open and preventing involuntary blinking during an ultrasound imaging procedure while ensuring patient safety and comfort. Eyelids can be taped up to the forehead or down to the cheek with common medical tape; however, this does not provide the instrument operator with the ability to adjust or control the amount of eye lid opening very well, nor allow the patient to relax the eyelids between scanning sessions. The present disclosure includes a speculum that can be placed in an eye piece such as used in a precision ultrasound device or other imaging device wherein the optical acoustic and transmission path between the eye and instrument is formed by a fluid such as saline solution and distilled water.

### CROSS REFERENCE TO RELATED APPLICATIONS

This application claims the benefits, under 35 U.S.C. § 119(e), of U.S. Provisional Application Ser. No. 63/044,857 entitled “A Method and Apparatus for Controlling an Eye Lid During Ultrasound Imaging” filed Jun. 26, 2020, which is incorporated herein by reference.

### FIELD OF INVENTION

The embodiment relates to a method and apparatus for holding an eyelid open and preventing involuntary blinking during an ultrasound imaging procedure while ensuring patient safety and comfort.

### BACKGROUND OF THE INVENTION

Ultrasonic imaging has found use in accurate and reproducible measurements of structures of the eye, such as, for example, the cornea and lens capsule. Such measurements provide an ophthalmic surgeon valuable information that can be used to guide various surgical procedures, such as LASIK and lens replacement, for correcting refractive errors. They also provide diagnostic information after surgery has been performed to assess the geometrical location of corneal features such as the LASIK scar and lens features such as lens connection, position, and orientation. This allows the surgeon to assess post surgical changes in the cornea or lens and to take steps to correct any problems that develop.

Except for on-axis measurements, dimensions, and locations of eye components behind the iris cannot be fully determined by optical means. Ultrasonic imaging in the frequency range of about 5 MHz to about 80 MHz can be applied to make accurate and precise measurements of structures of the eye, such as the cornea, lens capsule, ciliary muscle, zonules and the like.

An arc scanner is an ultrasound scanning device utilizing a transducer that both sends and receives pulses as it moves along an arcuate guide track. The arcuate guide track has a center of curvature whose position can be moved to scan different curved surfaces. Later versions of arc scanners have mechanisms that allow the radius of curvature of the scanner to be changed. In this type of scanner, a transducer is moved along an arcuate guide track whose center of curvature can be changed and set approximately at the center of curvature of the eye surface of interest. The transducer generates many acoustic pulses as it moves along the arcuate guide track. These pulses reflect off specular surfaces and other tissue interfaces within the eye. Each individual return pulse is detected and digitized to produce a series of A-scans. The A-scans can then be combined to form a cross-sectional image of the eye features of interest. The image combining A-scans is commonly called a B scan.

At a center frequency of about 38 MHz, a typical arc scanner has an axial resolution of about 20 microns and a lateral resolution of about 150 microns. The reproducibility of arc scanner images is typically about 2 microns.

Ultrasonic imaging requires a liquid medium to be interposed between the object being imaged and the transducer, which requires, in turn, that the eye, the transducer and the path between them be at all times immersed in a liquid medium. Concern for safety of the cornea introduces the practical requirement that the liquid medium be either pure water or normal saline water solution. There are reasons to prefer that the medium be pure water or physiologic saline (also known as normal saline) but the embodiments do not exclude other media suitable for conducting acoustic energy in the form of ultrasound. Most other media present an increased danger to the patient's eye, even with a barrier interposed between the eye and the ultrasonic transducer. Barriers can leak or be breached, allowing the liquids on either side to mix, thus bringing a potentially harmful material into contact with the eye.

An eyepiece serves to complete a continuous acoustic path for ultrasonic scanning, that path extending from the transducer to the surface of the patient's eye. The eyepiece also separates the water in which the patient's eye is immersed from the water in the chamber in which the ultrasound transducer and guide track assembly are contained. Finally, the eyepiece provides a steady rest for the patient and helps the patient to remain steady during a scan. To be practical, the eyepiece should be free from frequent leakage problems, should be comfortable to the patient and its manufacturing cost should be low since it should be replaced for every new patient.

Another ultrasound scanning method is known as Ultrasound Bio Microscopy as embodied in a hand-held device commonly known as a UBM. A UBM can capture anterior segment images using a transducer to emit short acoustic pulses ranging from about 20 to about 80 MHz. This type of ultrasound scanner is also called a sector scanner.

A UBM is a hand-held ultrasonic scanner whose beams sweep a sector like a radar. The swept area is pie-shaped with its central point typically located near the face of the ultrasound transducer. In this type of scanner, an ultrasonic transducer oscillates about a fixed position so as to produce many acoustic echoes which are captured as a series of A-scans. These A-scans can then be combined to form a B-scan of a localized region of interest within the eye.

The UBM method is capable of making qualitative ultrasound images of the anterior segment of the eye but cannot make accurate, precision, comprehensive, measurable images of the cornea, lens or other components of the eye required for glaucoma screening, keratoconus evaluation or lens sizing. This is because of two reasons. First, the UBM is a hand-held device and relies on the steadiness of the operator's hand to maintain a fixed position relative to the eye being scanned for several seconds. Second, the UBM is pressed firmly onto the patient's eye to make contact with the patient's cornea to obtain good acoustic coupling. This gives rise to some distortion of the cornea and the eyeball.

Between these two limitations, the resolution is limited, at best, to the range of 40 to 60 microns and the reproducibility, at best, can be no better than 20 microns.

Optical Coherence Tomography (OCT) is a light-based imaging technology that can image the cornea although not to the full lateral extent as can an ultrasound instrument. OCT cannot see behind the scleral wall or behind the iris and is therefore of limited use in glaucoma screening. OCT does well for imaging the central retina although only to the lateral extent allowed by a dilated pupil. OCT images of the retina can disclose the damage caused by glaucoma. The approach of a precision ultrasound scanning device is to detect the onset of glaucoma by imaging structural changes in the anterior segment before any retinal damage occurs so that the disease can be identified and successfully treated with drugs and/or stents.

Ultrasonic imaging has been used in corneal procedures such as LASIK to make accurate and precise images and maps of cornea thickness which include epithelial thickness, Bowman's layer thickness and images of LASIK flaps.

New procedures such as implantation of accommodative lenses may provide nearly perfect vision without spectacles or contact lenses. Implantation of accommodative lenses requires precision measurements of, for example, the position and width of the natural lens for successful lens powering and implantation. Ultrasonic imaging can be used to provide the required accurate images of the natural lens especially where the zonules attach the lens to the ciliary body which is well off-axis and behind the iris and therefore not accessible to optical imaging.

Recent advances in ultrasonic imaging have allowed images of substantially the entire lens capsule to be made. This has opened up the ability of diagnostic devices to assist in both research of lens implantation devices and strategies, and to planning, executing and follow-up diagnostics for corrective lens surgery including specialty procedures such as glaucoma and cataract treatments as well as implantation of clear intraocular lenses including accommodative lens.

A phakic intraocular lens (PIOL) is a special kind of intraocular lens that is implanted surgically into the eye to correct myopia. It is called “phakic” (meaning “having a lens”) because the eye's natural lens is left untouched. Intraocular lenses that are implanted into eyes after the eye's natural lens has been removed during cataract surgery are known as pseudophakic. Phakic intraocular lenses are considered for patients with high refractive errors when laser options, such as LASIK and PRK are not the best surgical options.

PIOLS made of collamer (a foldable gel-like substance) requires a very small incision due the flexibility of the material. In the cases where refractive outcomes are not the best, LASIK can be used for fine-tuning. If a patient eventually develops a visually significant cataract, the PIOLs can be removed (explanted) when the patient undergoes cataract surgery.

### Speed of Sound in Different Regions of an Eye

Both ultrasound sector and ultrasound arc scanning instruments record time-of-arrival of reflected ultrasound pulses. A speed of sound of the medium is then used to convert these time of arrival measurements to distance measurements. Traditionally, a single representative speed of sound value is used. Usually the speed of sound of water at 37 C (1,531 m/s) is used although speeds of sound from 1,531 m/s to 1,641 m/s may be used (1,641 m/s is the speed of sound in a natural human lens).

The speed of sound varies in the different anterior segment regions of the eye such as the cornea, aqueous, natural lens, and vitreous fluid. The speed of sound in these different regions have been measured by various researchers and are reasonably known. Therefore if the interfaces of these regions can be identified, the appropriate speeds of sounds for these regions can be used to convert times of arrivals to distances with more accuracy.

### Unintended Eye Motion and Instrument Motion During Scanning

It is also important to compensate for unintended patient head or eye motion because a scan of the anterior segment scan or lens capsule scan is typically made by overlaying two or three separate scans (such as an arcuate scan followed by two linear scans, also described in U.S. Pat. No. 9,597,059 entitled “Tracking Unintended Eye Movements in an Ultrasonic Scan of the Eye”.

Unintended patient eye motion includes saccades which are quick, simultaneous rotations of both eyes in the same direction involving a succession of discontinuous individual rotations of the eye orbit in the eye socket.

The speed of transducer motion in an precision scanning device such as described, for example, in U.S. Pat. No. 8,317,709, is limited because its movement is in a bath of water and excessive speed of motion of the transducer and its carriage can result in vibration of the entire instrument. In practice, a set of ultrasound scans can be carried out in about 1 to about 3 minutes from the time the patient's eye is immersed in water to the time the water is drained from the eyepiece.

The actual scanning process itself can be carried out in several tens of seconds, after the operator or automated software completes the process of centering and range finding. As is often the case, the patient may move his or her head slightly or may move his or her eye in its socket during this time. In some cases, the patient's heartbeat can be detected as a slight blurring of the images. If patient movements are large, the scan set can always be repeated.

### Creating Composite B-Scans

The arc scanning instrument of the present disclosure can create several distinct scan types. These are:
an arcuate scan having a fixed radius of curvature.
a linear scan
a combined arcuate and linear scan allowing for various radii of curvature including inverse radii of curvature.

These scans can be combined to form composite images because each image is formed from very accurate time-of-arrival data and transducer positional data. However, combining these separate scans into a composite scan must take into account patient eye movement during scanning; and instrument movement during scanning.

Due to the need for an eyes seal to provide a continuous medium for the ultrasound signal to travel between the transducer, any scanning device has a limitation in the range of movement the transducer can make relative to the eye. The range of the scanning device can be expanded to cover more of the anterior segment by introducing intentional and controlled eye movements and scanning the newly exposed portion of the eye that can now be reached. Registration techniques can be used to combine the scans of different eye positions to create a more complete composite image of the anterior segment of the eye.

U.S. patent application Ser. No. 16/422,182 entitled “Method for Measuring Behind the Iris after Locating the Scleral Spur” is pending. This application is directed towards a method for locating the scleral spur in an eye using a precision ultrasound scanning device for imaging of the anterior segment of the eye. One of the applications of a precision ultrasound scanning device or instrument is to image the region of the eye where the cornea, iris, sclera, and ciliary muscle are all in close proximity. By using a knowledge of the structure of the eye in this region and employing binary filtering techniques, the position of the scleral spur can be determined. Once the position of the scleral spur is determined, a number of measurements that characterize the normal and abnormal shapes of components within this region of the anterior segment of the eye can be made.

Ultrasonic imaging has found use in accurate and reproducible measurements of structures of the eye, such as, for example, the cornea and lens capsule. Such measurements provide an ophthalmic surgeon valuable information that can be used to guide various surgical procedures for correcting refractive errors such as LASIK and lens replacement. They also provide diagnostic information after surgery has been performed to assess the geometrical location of corneal features such as the LASIK scar and lens features such as lens connection, position, and orientation. This allows the surgeon to assess post surgical changes in the cornea or lens and to take steps to correct any problems that develop.

Except for on-axis measurements, dimensions, and locations of eye components behind the iris cannot be fully determined by optical means. Ultrasonic imaging in the frequency range of about 5 MHz to about 80 MHz can be applied to make accurate and precise measurements of structures of the eye, such as the cornea, lens capsule, ciliary muscle, and the like.

### A Remaining Problem

An ultrasonic scan of the eye may include one or more rapid B-scans (each B-scan formed from a plurality of A-scans) at each of several meridians (typically about 3 to about 12 meridians) and these may be combined automatically to form a comprehensive image of the anterior segment. Therefore it is necessary to rapidly scan a patient to reduce the possibility of patient eye motion during a scan session. Further, it may be necessary to re-scan a patient at a later time in order to determine if changes in features or dimensions has occurred.

It is also important to compensate for unintended patient head or eye motion because a scan of the anterior segment scan or lens capsule scan is typically made by overlaying two or three separate scans (such as an arcuate scan followed by two linear scans, also described in U.S. Pat. No. 9,597,059 entitled “Tracking Unintended Eye Movements in an Ultrasonic Scan of the Eye”.

The speed of transducer motion in an precision scanning device such as described, for example, in U.S. Pat. No. 8,317,709, is limited because its movement is in a bath of water and excessive speed of motion of the transducer and its carriage can result in vibration of the entire instrument. In practice, a set of ultrasound scans can be carried out in about 1 to about 3 minutes from the time the patient's eye is immersed in water to the time the water is drained from the eyepiece. The actual scanning process itself can be carried out in several tens of seconds, after the operator or automated software completes the process of centering and range finding. As is often the case, the patient may move his or her head slightly or may move his or her eye in its socket during this time. In some cases, the patient's heartbeat can be detected as a slight blurring of the images. If patient movements are large, the scan set can always be repeated.

Imaging of the internal anatomy of the eye requires some form of energy to enter the eye and be reflected back to a transducer for detection. Ultrasound is one such type of energy, as is light. A common challenge during these procedures is to keep the eyelids from blocking the incoming or returning energy either due to the natural position of the eyelids or during blinking by the patient. The present disclosure provides a means for preventing an eyelid from closing and/or to retract an eyelid further beyond its natural location to increase the range and space available for the imaging system.

The present disclosure is directed to a method and apparatus for holding an eyelid open and preventing involuntary blinking during an ultrasound imaging procedure while ensuring patient safety and comfort. Eyelids can be taped up to the forehead or down to the cheek with common medical tape; however, this does not provide the instrument operator with the ability to adjust or control the amount of eye lid opening very well, nor allow the patient to relax the eyelids between scanning sessions. This concept of eyelids taped up to the forehead or down to the cheek with common medical tape is disclosed in U.S. patent application Ser. No. 17/133,233 entitled “A Method and Apparatus for Controlling an Eye Lid During Imaging” filed Dec. 23, 2020, which is incorporated herein by reference.

An eye speculum, installed under a patient's eye lid, can also be used for the described purpose; however, an eye speculum does not allow for control by the patient or instrument operator either. An eye speculum is often quite uncomfortable for some patients.

There remains, therefore, a need for a method and apparatus that can be used to hold an eyelid open and prevent involuntary blinking during an ultrasound imaging procedure that can be integrated into the eye piece. A speculum that is compatible with an eye piece such as used in a precision ultrasound device is disclosed herein can fill this requirement while ensuring patient safety and comfort.

### SUMMARY OF THE INVENTION

These and other needs are addressed by the present disclosure. The various embodiments and configurations of the present disclosure are directed generally to ultrasonic imaging of biological materials such as the cornea, sclera, iris, and lens in the anterior segment of an eye and in particular directed to a method for holding an eyelid open and preventing involuntary blinking during an ultrasound imaging procedure.

The present disclosure consists of a plastic speculum that can be inserted in a patient's eye and then the patient can place his or her eye against an eyepiece attached to a precision ultrasound imaging device. The eye piece can then be filled with saline solution to allow good acoustic coupling between the patient's eye and the imaging ultrasound transducer.

The speculum of the present disclosure is approximately 1.7 inches long by approximately 1.05 inches wide by approximately 0.7 inches tall.

The speculum itself can be manufactured in several ways, including but not limited to injection molding, 3D printing, vacuum forming, polymer casting and extrusion.

As discussed below, the speculum can be used for ultrasound imaging using an arc scanner device and its disposable eye piece.

In this disclosure, a speculum is described comprising opposing pads further comprising an adhesive to adhere to skin of a patient; and a flexible eyelid retraction member engaging the opposing pads. The retraction member comprises a plurality of curved surfaces collectively sized to surround an eye of a patient and to press outwardly against the eyelids of the patient to urge the upper and lower eyelids to retract from the eye. The speculum of the present disclosure is received by an eye seal of an ocular imaging device such as other imaging field of view devices such as OCT instruments, topography devices such the OCULUS Pentacam, and measurement devices such as tonometers and the like.

The following definitions are used herein:

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The phrases at least one, one or more, and and/or are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

An acoustically reflective surface or interface is a surface or interface that has sufficient acoustic impedance difference across the interface to cause a measurable reflected acoustic signal. A specular surface is typically a very strong acoustically reflective surface.

Anterior means situated at the front part of a structure; anterior is the opposite of posterior.

An A-scan is a representation of a rectified, filtered reflected acoustic signal as a function of time, received by an ultrasonic transducer from acoustic pulses originally emitted by the ultrasonic transducer from a known fixed position relative to an eye component.

Accuracy as used herein means substantially free from measurement error.

Aligning means positioning the acoustic transducer accurately and reproducibly in all three dimensions of space with respect to a feature of the eye component of interest (such as the center of the pupil, center of curvature or boundary of the cornea, lens, retina, etcetera).

The anterior chamber comprises the region of the eye from the cornea to the iris.

The anterior segment comprises the region of the eye from the cornea to the back of the lens.

Automatic refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Auto-centering means automatically, typically under computer control, causing centration of the arc scanning transducer with the eye component of interest.

A B-scan is a processed representation of A-scan data by either or both of converting it from a time to a distance using acoustic velocities and by using grayscales, which correspond to A-scan amplitudes, to highlight the features along the A-scan time history trace (the latter also referred to as an A-scan vector).

A canthus is the angular junction of the eyelids at either corner of the eye where the upper and lower eyelids meet.

Centration means substantially aligning the center of curvature of the arc scanning transducer in all three dimensions of space with the center of curvature of the eye component of interest (such as the cornea, pupil, lens, retina, etcetera) such that rays from the transducer pass through both centers of curvature. A special case is when both centers of curvature are coincident.

The ciliary body is the circumferential tissue inside the eye composed of the ciliary muscle and ciliary processes. There are three sets of ciliary muscles in the eye, the longitudinal, radial, and circular muscles. They are near the front of the eye, above and below the lens. They are attached to the lens by connective tissue called the zonule of Zinn, and are responsible for shaping the lens to focus light on the retina. When the ciliary muscle relaxes, it flattens the lens, generally improving the focus for farther objects. When it contracts, the lens becomes more convex, generally improving the focus for closer objects.

Coronal means of or relating to the frontal plane that passes through the long axis of a body. With respect to the eye or the lens, this would be the equatorial plane of the lens which also approximately passes through the nasal canthus and temporal canthus of the eye.

An eye speculum is an instrument or device for keeping the eyelids apart during an inspection of or a procedure on or an operation on the eye. Examples are made from plated steel wire or plastic. Luer's, Von Graefe's, and Steven's are the most common types.

Fiducial means a reference, marker or datum in the field of view of an imaging device.

Fixation means having the patient focus an eye on an optical target such that the eye's optical axis is in a known spatial relationship with the optical target. In fixation, the light source is axially aligned in the arc plane with the light source in the center of the arc so as to obtain maximum signal strength such that moving away from the center of the arc in either direction results in signal strength diminishing equally in either direction away from the center.

A guide or guide track e is an apparatus for directing the motion of another apparatus.

Haptics are little protrusions extending from the outer diameter of some types of artificial lenses. These haptics fix the position of the lens to the ciliary body by protruding into the ciliary sulcus. In the case of accommodative lenses, the haptics enable the lens to accommodate in response to the action of the ciliary body.

The home position of the imaging ultrasound transducer is its position during the registration process.

An intraocular lens is an artificial lens that is implanted in the eye to take the place of the natural lens.

As used herein, a meridian is a 2-dimensional plane section through the approximate center of a 3-dimensional eye and its angle is commonly expressed relative to a horizon defined by the nasal canthus and temporal canthus of the eye.

The natural lens (also known as the aquila or crystalline lens) is a transparent, biconvex structure in the eye that, along with the cornea, helps to refract light to be focused on the retina. The lens, by changing shape, functions to change the focal distance of the eye so that it can focus on objects at various distances, thus allowing a sharp real image of the object of interest to be formed on the retina. This adjustment of the lens is known as accommodation. The lens is located in the anterior segment of the eye behind the iris. The lens is suspended in place by the zonular fibers, which attach to the lens near its equatorial line and connect the lens to the ciliary body. The lens has an ellipsoid, biconvex shape whose size and shape can change due to accommodation and due to growth during aging. The lens is comprised of three main parts: namely the lens capsule, the lens epithelium, and the lens fibers. The lens capsule forms the outermost layer of the lens and the lens fibers form the bulk of the interior of the lens. The cells of the lens epithelium, located between the lens capsule and the outermost layer of lens fibers, are generally found only on the anterior side of the lens.

Ocular means having to do with the eye or eyeball.

Ophthalmology means the branch of medicine that deals with the eye.

Optical as used herein refers to processes that use light rays.

The optical axis of the eye is a straight line through the centers of curvature of the refracting surfaces of an eye (the anterior and posterior surfaces of the cornea and lens).

As used herein, the orbit of the eye is the cavity or socket of the skull in which the eye and its appendages are situated. In the adult human, the volume of the orbit is about 30 ml, of which the eye occupies about 6.5 ml.

Phakic intraocular lenses, or phakic lenses, are lenses made of plastic or silicone that are implanted into the eye permanently to reduce a person's need for glasses or contact lenses. Phakic refers to the fact that the lens is implanted into the eye without removing the eye's natural lens. During phakic lens implantation surgery, a small incision is normally made in the front of the eye. The phakic lens is inserted through the incision and placed just in front of or just behind the iris.

Positioner means the mechanism that positions a scan head relative to a selected part of an eye. In the present disclosure, the positioner can move back and forth along the x, y or z axes and rotate in the β direction about the z-axis. Normally the positioner does not move during a scan, only the scan head moves. In certain operations, such as measuring the thickness of a region, the positioner may move during a scan.

Position tracking sensors are a set of position sensors whose sole purpose is to monitor the movement of the eye or any other anatomical feature during the imaging scan so as to remove unwanted movement of the feature.

Posterior means situated at the back part of a structure; posterior is the opposite of anterior.

The posterior chamber comprises the region of the eye from the back of the iris to the front of the lens.

The posterior segment comprises the region of the eye from the back of the lens to the rear of the eye comprising the retina and optical nerve.

Precise as used herein means sharply defined and repeatable.

Precision means how close in value successive measurements fall when attempting to repeat the same measurement between two detectable features in the image field. In a normal distribution precision is characterized by the standard deviation of the set of repeated measurements. Precision is very similar to the definition of repeatability.

The pulse transit time across a region of the eye is the time it takes a sound pulse to traverse the region.

Refractive means anything pertaining to the focusing of light rays by the various components of the eye, principally the cornea and lens.

Registration as used herein means aligning.

Saccades are quick, simultaneous rotations of both eyes in the same direction involving a succession of discontinuous individual rotations of the eye orbit in the eye socket. These rapid motions can be on the order of 20 degrees of rotation with a maximum velocity of 200 degrees/sec and are a part of normal eyesight.

Scan head means the mechanism that comprises the ultrasound transducer, the transducer holder and carriage as well as any guide tracks that allow the transducer to be moved relative to the positioner. Guide tracks may be linear, arcuate or any other appropriate geometry. The guide tracks may be rigid or flexible. Normally, only the scan head is moved during a scan.

Sector scanner is an ultrasonic scanner that sweeps a sector like a radar. The swept area is pie-shaped with its central point typically located near the face of the ultrasound transducer.

A specular surface means a mirror-like surface that reflects either optical or acoustic waves. For example, an ultrasound beam emanating from a transducer will be reflected directly back to that transducer when the beam is aligned perpendicular to a specular surface.

Surgical eye specula or ophthalmic speculums are used for retracting and holding the eyelid during surgery. Ophthalmic speculums include wire speculums, screw speculums, pinion speculums, spring speculums, and aspiration speculums.

The ciliary sulcus is the groove between the iris and ciliary body. The scleral sulcus is a slight groove at the junction of the sclera and cornea.

A thermoplastic, or thermosoftening plastic, is a plastic polymer material that becomes pliable or moldable at a certain elevated temperature and solidifies upon cooling.

A track or guide track is an apparatus along which another apparatus moves. In an ultrasound scanner or combined ultrasound and optical scanner, a guide track is an apparatus along which one or more ultrasound transducers and/or optical probes moves during a scan.

Ultrasonic means sound that is above the human ear's upper frequency limit. When used for imaging an object like the eye, the sound passes through a liquid medium, and its frequency is many orders of magnitude greater than can be detected by the human ear. For high-resolution acoustic imaging in the eye, the frequency is typically in the approximate range of about 5 to about 80 MHz.

An ultrasonic scanner is an ultrasound scanning device utilizing a transducer that both sends and receives pulses as it moves along 1) an arcuate guide track, which guide track has a center of curvature whose position can be moved to scan different curved surfaces; 2) a linear guide track; and 3) a combination of linear and arcuate guide tracks which can create a range of centers of curvature whose position can be moved to scan different curved surfaces.

The visual axis of the eye is the line joining the object of interest and the fovea and which passes through the nodal points of the eye.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein. By way of example, the phrase from about 2 to about 4 includes the whole number and/or integer ranges from about 2 to about 3, from about 3 to about 4 and each possible range based on real (e.g., irrational and/or rational) numbers, such as from about 2.1 to about 4.9, from about 2.1 to about 3.4, and so on.

The preceding is a simplified summary of the disclosure to provide an understanding of some aspects of the disclosure. This summary is neither an extensive nor exhaustive overview of the disclosure and its various embodiments. It is intended neither to identify key or critical elements of the disclosure nor to delineate the scope of the disclosure but to present selected concepts of the disclosure in a simplified form as an introduction to the more detailed description presented below. As will be appreciated, other embodiments of the disclosure are possible utilizing, alone or in combination, one or more of the features set forth above or described in detail below.

### BRIEF DESCRIPTION OF THE DRAWINGS

The present disclosure may take form in various components and arrangements of components, and in various steps and arrangements of steps. The drawings are only for purposes of illustrating the preferred embodiments and are not to be construed as limiting the disclosure. In the drawings, like reference numerals may refer to like or analogous components throughout the several views.

FIGS. 1a-b show various types of prior art ophthalmic speculums.

FIG. 2 shows an alternate method to hold an eyelid open during imaging (from U.S. patent application Ser. No. 17/133,233 filed Dec. 23, 2020, which is incorporated herein by reference).

FIG. 3 is a schematic of a prior art Insight 100 ultrasound imaging device for the eye.

FIG. 4 is a schematic of a prior art eye piece used on the Insight 100.

FIG. 5 is a cut-away of a prior art Insight 100 with a patient in position for imaging.

FIG. 6 is a prior art schematic showing the relationship between the scan head, the ultrasound transducer, the eye seal and the patient's eye during imaging.

FIG. 7 is a schematic of the speculum of the embodiment as it would fit inside an eye piece.

FIG. 8 is another schematic of the speculum of the embodiment as it would fit inside an eye piece.

FIG. 9 is a schematic of the speculum of the embodiment.

FIGS. 10a-f show various schematic views of the speculum of the embodiment.

FIG. 11 illustrates approximate cost versus volume for several plastic fabrication processes.

FIGS. 12a-f show schematic views of various speculum applicator tool designs of the embodiment.

FIG. 13 is a schematic of a speculum applicator tool clamping on a speculum of the embodiment.

### DETAILED DESCRIPTION OF THE DRAWINGS

### Prior Art

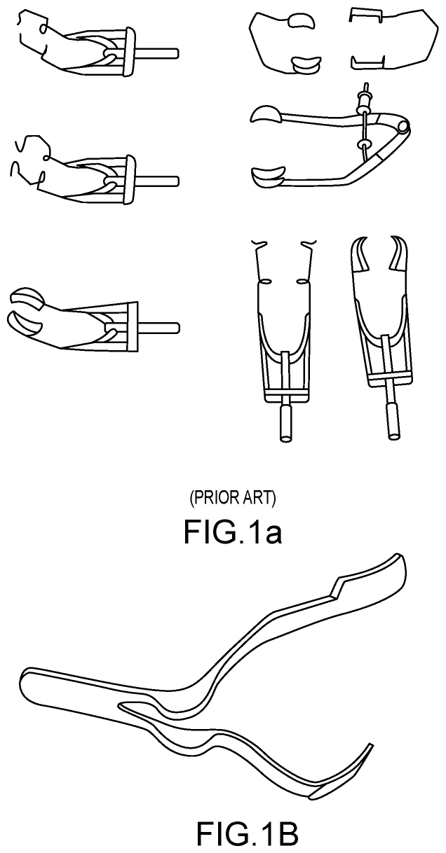

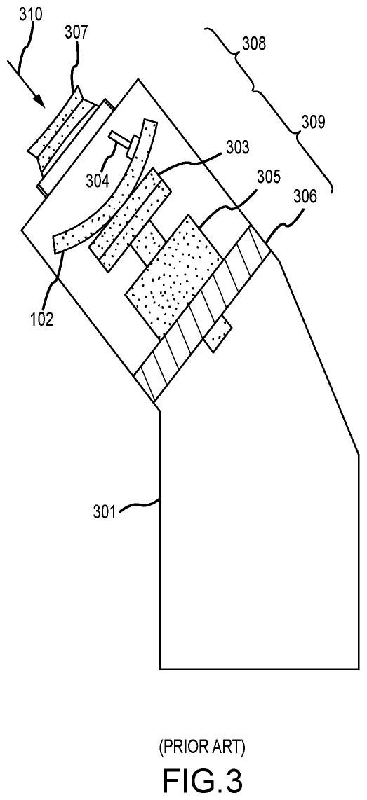

FIG. 1 shows various types of prior art ophthalmic speculums. FIG. 1a shows a number of types of eye speculums that have been used. None of these would be useful to hold open eye lids during imaging with the ultrasound scanner such as shown in FIG. 3. Adjustments of these speculums could not be made by the patient. FIG. 1b shows a molded plastic speculum taken from U.S. D601,6985 “Eye Speculum”. This speculum can be used to hold open eye lids during imaging with the ultrasound scanner such as shown in FIG. 3. This speculum causes some patient discomfort but cannot be adjusted by the instrument operator or patient once the patient is engaged with the eye piece of the scanning instrument shown in FIG. 3.

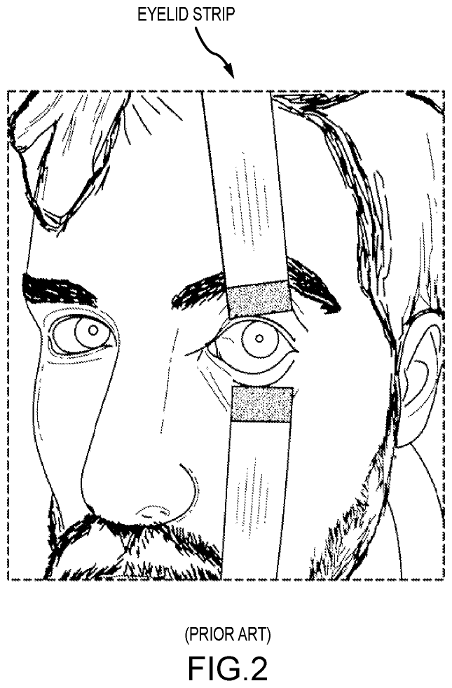

FIG. 2 shows an alternate method to hold an eyelid open during imaging. FIG. 2 is a photo of an eye strip applied to a patient prior to imaging. One assembled eye strip is attached to the upper eye lid using the short section of exposed adhesive on the one sided tape. A second assembled eye strip is attached to the lower eye lid using the short section of exposed adhesive on its one sided tape. The patient pulls their own eye lid open then positions himself at the scanning machine.

Eyelids can be taped up to the forehead or down to the cheek with common medical tape; however, this does not provide the instrument operator with the ability to adjust or control the amount of eye lid opening very well, nor to relax the eyelids, for instance between scanning, and then reapply the tension. This concept is disclosed in U.S. patent application Ser. No. 17/133,233 entitled “A Method and Apparatus for Controlling an Eye Lid During Imaging” filed Dec. 23, 2020, which is incorporated herein by reference.

### Ultrasound Imaging Instrument

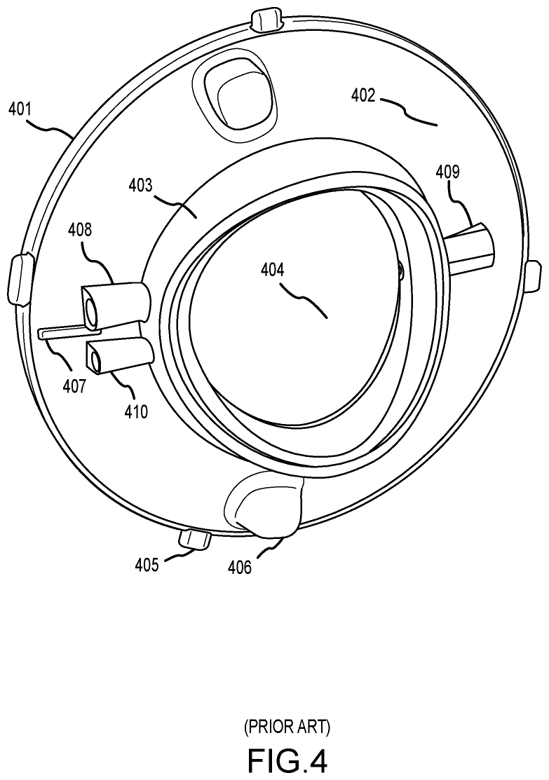

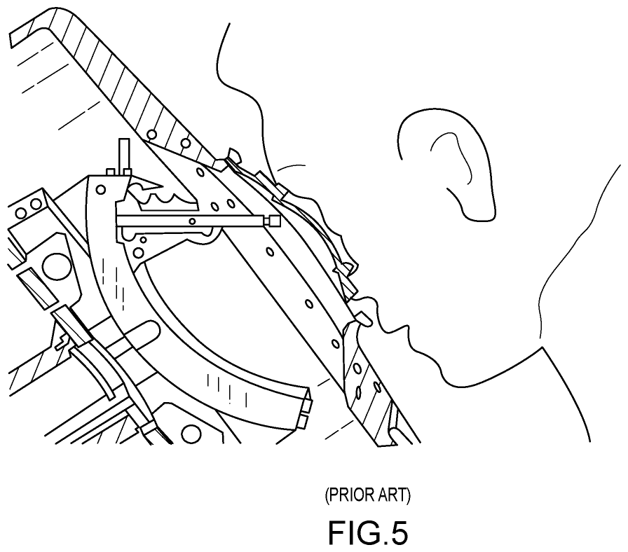

FIG. 3 is a prior art schematic of an Insight 100 ultrasound imaging device for the eye. FIG. 3 is a schematic of the principal elements of a prior art ultrasound eye scanning device such as described in U.S. Pat. No. 8,317,709, entitled “Alignment and Imaging of an Eye with an Ultrasonic Scanner”. The scanning device 301 of this example is comprised of a disposable eyepiece 307, a scan head assembly 308 and a positioning mechanism 309. The scan head assembly 308 is comprised of an arcuate guide 302 with a scanning transducer 304 on a transducer carriage which moves back and forth along the arcuate guide track 302, and a linear guide track 303 which moves the arcuate guide track 302 back and forth (as described further in FIG. 3). The positioning mechanism 309 is comprised of an x-y-z and beta mechanisms 305 (described in FIG. 4) mounted on a base 306. The base 306 is rigidly attached to the scanning device 301. A longitudinal axis 310 passes generally through a center of the head assembly 308 and is substantially perpendicular to a face of the eyepiece 307. A video camera (not shown) may be positioned within the scanning device 301 and aligned with the longitudinal axis 310 to provide an image of a patient's eye through the eyepiece 307. The scanning device 301 is typically connected to a computer (not shown) which includes a processor module, a memory module, a keyboard, a mouse or other pointing device, a printer, and a video monitor. One or more fixation lights (not shown) may be positioned within the scanning device at one or more locations. The eyepiece 307 may be disposable as described in FIG. 5.

The positioner assembly 309 and scan head assembly 308 are both fully immersed in water (typically distilled water) which fills the chamber from base plate 306 to the top of the chamber on which the eyepiece 307 is attached.

A patient is seated at the scanning device 301 with one eye engaged with the disposable eyepiece 307. The patient is typically directed to look downward at one of the fixation lights during a scan sequence. The patient is fixed with respect to the scanning device 301 by a headrest system such as shown, for example, in FIG. 4, and by the eyepiece 307.

An operator using a mouse and/or a keyboard and the video monitor, for example, inputs information into the computer selecting the type of scan and scan sequences as well as the desired type of output analyses. The operator using the mouse and/or the keyboard, the video camera located in the scanning machine, and the video screen, centers a reference marker such as, for example, a set of cross hairs displayed on the video screen on the desired component of the patient's eye which is also displayed on video screen. This is done by setting one of the cross hairs as the prime meridian for scanning. These steps are carried out using the positioning mechanism which can move the scan head in the x, x, z and beta space (three translational motions plus rotation about the z-axis). The z-axis is parallel to the longitudinal axis 310. Once this is accomplished, the operator instructs the computer to proceed with the scanning sequence. Now the computer processor takes over the procedure and issues instructions to the scan head 308 and the scanning transducer 304 and receives positional and imaging data. The computer processor proceeds with a sequence of operations such as, for example: (1) with the transducer carriage substantially centered on the arcuate guide track, rough focusing of the scanning transducer 304 on a selected eye component; (2) accurately centering of the arcuate guide track with respect to the selected eye component; (3) accurately focusing the scanning transducer 304 on the selected feature of the selected eye component; (4) rotating the scan head assembly 308 through a substantial angle (including orthogonal) and repeating steps (1) through (3) on a second meridian; (5) rotating the scan head back to the prime meridian; (6) initiating a set of A-scans along each of the of selected scan meridians, storing this information in the memory module; (7) utilizing the processor, converting the A-scans for each meridian into a set of B-scans and then processing the B-scans to form an image associated with each meridian; (8) performing the selected analyses on the A-scans, B-scans and images associated with each or all of the meridians scanned; and (9) outputting the data in a preselected format to an output device such as a printer. As can be appreciated, the patient's head must remain fixed with respect to the scanning device 301 during the above operations when scanning is being carried out, which in a modern ultrasound scanning machine, can take several tens of seconds.

An eyepiece serves to complete a continuous acoustic path for ultrasonic scanning, that path extending in water from the transducer to the surface of the patient's eye. The eyepiece 307 also separates the water in which the patient's eye is immersed (typically a saline solution) from the water in the chamber (typically distilled water) in which the transducer guide track assemblies are contained. The patient sits at the machine and looks down through the eyepiece 307 in the direction of the longitudinal axis 310. Finally, the eyepiece provides an additional steady rest for the patient and helps the patient's head to remain steady during a scan procedure.

### Eye Piece for Ultrasound Imaging

FIG. 4 is a prior art schematic of an eye piece typically used on an ultrasound scanner such as the Insight 100. FIG. 4 is an isometric view of an advanced eye piece for a precision scanning machine. Eye piece 401 is comprised of a plastic base 402 molded from a plastic such as ABS and a soft rubber conformable face seal 403 formed from a silicone thermo-plastic elastomer. The conformable face seal 403 is over-molded onto the plastic base 402 by a heat process typically applied to the conformable face seal 403. Plastic base 402 also includes attaching mechanisms 405 which attach the eye piece to the mounting ring (not shown) which is typically attached to the main scanner housing; thumb and finger protrusions 406 used to rotate the eye piece into the mounting ring; indexing ridge 407 which prevents over-rotation of the eye piece as it is rotated into the mounting ring attached to the main scanner housing; and fill port 408, vent port 410 and drain port 409. Ports 408, 409 and 410 allow fluid flow through the eye piece base 401.

The eye piece is attached and sealed to a mounting ring which is, in turn, attached to the main scanner body by a groove molded as part of the eye piece base 402 and a matching tongue formed as part of the mounting ring. The eye piece is rotated into position with the mounting ring where the tongue and groove form a contact connection which compresses and seals as the parts are rotated into position.

A sealed hygienic barrier membrane (not shown) is formed as part of the eye piece and is typically located, where the soft rubber face seal 403 is connected to the eye piece base 401. This membrane is typically attached onto the plastic eye piece base 402 by an adhesive backing commonly used in medical disposable components. The thickness of the membrane is designed for transmission of light (such as a fixation light, and transmission of acoustic energy emitted by the transducer and reflected by a component of the eye. The membrane is hermetically sealed to prevent saline solution from contaminating the distilled water in the machine body (saline solution or tap water inside the machine body can corrode plastic, ceramic and metal components) and to prevent the distilled water in the machine body from contaminating the saline solution in the eye piece. As disclosed in U.S. Pat. No. 8,758,252, eye piece membranes have been made from materials such as, for example, polyethylene, mylar, polypropylene; vinylidene chloride; polyvinylidene chloride; or DuraSeal (made by Diversified Biotech) which is polyethylene based material free of adhesives. A preferred material is medical grade polyethylene which has an acoustic impedance slightly higher than that of water (about 2.33 million kg/m²-s compared to 1.54 million kg/m²-s for water). The thickness of the membrane is preferably in the range of about 10 to about 30 microns. This thickness is a small part of an acoustic wavelength in water which is about 150 microns at 10 MHz and about 20 microns at 80 MHz.

The fill, drain and vent ports shown in FIG. 4 are designed and sized for fast fill (to minimize the patient's time with their eye immersed in the saline solution), for venting of any bubbles that may form, for example, if the seal on the patient's head leaks or the patient pulls away from the machine, and for rapid draining of the saline solution back into the plastic saline bag after scanning is completed. As can be appreciated, the fill and vent ports are on the top of the eye piece and the drain port is on the bottom of the eye piece.

### Ultrasound Imaging Instrument Operation

FIG. 5 is a prior art cut-away of an Insight 100 with a patient in position for imaging. FIG. 5 is a rendering of an ultrasound scanner and patient being imaged. In this rendering, the bucket or compartment which holds the positioner and scan head assemblies and the water used during scanning, is shown in a cutaway view. This cutaway view also shows the ultrasound transducer with the probe tip very close to one side of the eye seal membrane and with the patient's eye on the other side of the membrane.

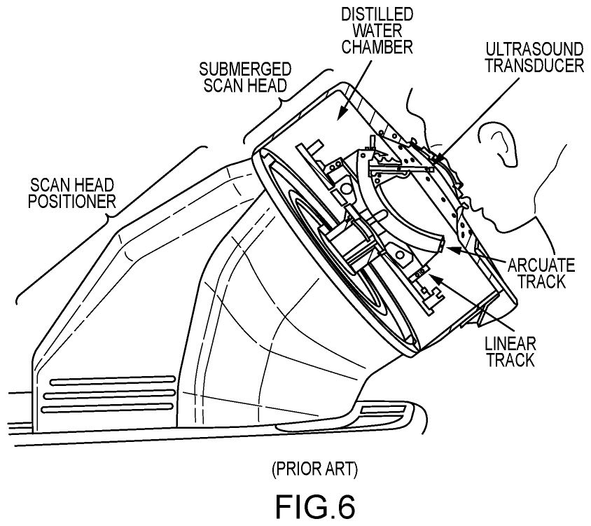

FIG. 6 is a prior art schematic showing the relationship between the scan head, the ultrasound transducer, the eye seal and the patient's eye during imaging. In this figure, an ultrasound transducer is shown as it moves along an arcuate guide track. As noted previously, the scan head and probe are immersed in scanner fluid (water) and a membrane contained by the eye piece or eye seal separates the scanner fluid from the saline solution in the eye seal cup. The cornea of the eye is immersed in the saline solution and the eye is sealed against a soft material, formed from a silicone thermo-plastic elastomer, that is part of the eye piece assembly. Thus the saline solution, the membrane and the scanner fluid form an acoustic path that has substantially the same acoustic impedance as the anterior segment components of the eye. The acoustic path is also optically transparent and allows an optical camera to assist in centering the eye just prior to scanning.

The scan head is mounted on a positioner mechanism. The positioner mechanism is fixed to the instrument body in the lower left of FIG. 6. The positioner includes a telescoping cylinder that goes through a flexible rubber seal that separates the lower compartment from the water chamber in which the scan head is located. The scan head is immersed in scanner fluid (typically distilled water) while the fixed section of the positioner mechanism is immersed in ambient air.

### Present Disclosure

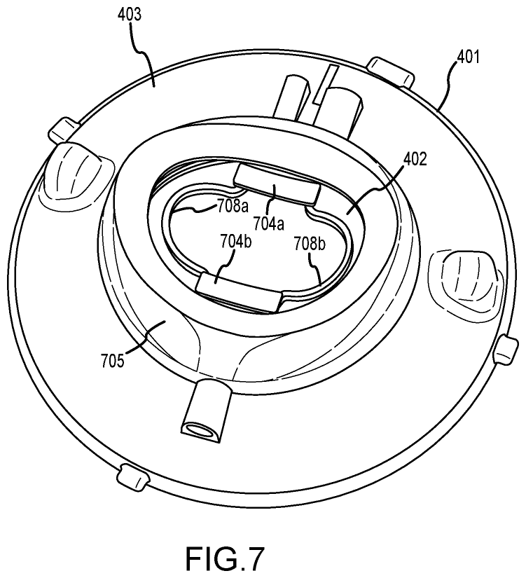

FIG. 7 is a schematic of the speculum of an embodiment as it would fit inside an eye piece 401. The speculum fits on the inner side of the sealed hygienic barrier membrane 402 that separates the saline solution into which the patient's eye is immersed from the distilled water in the imaging machine. The speculum comprises opposing pads 704a and 704b optionally comprising an adhesive to adhere to skin of a patient and a flexible eyelid retraction member 708a and 708b engaging the opposing pads 704a and 704b.

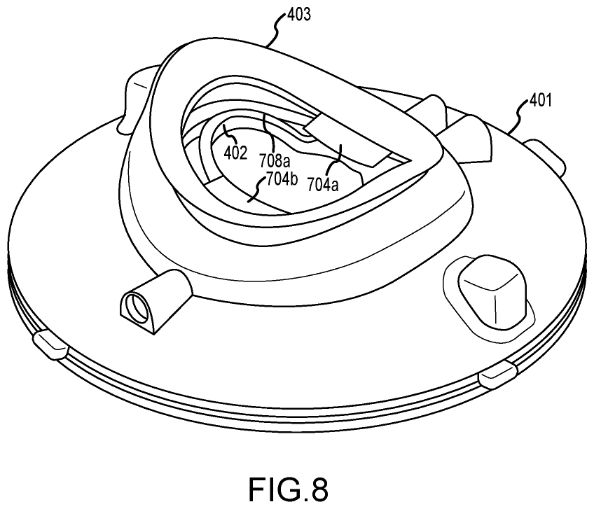

FIG. 8 is another schematic of the speculum of the embodiment as it would fit inside an eye piece. The speculum fits on the inner side of the sealed hygienic barrier membrane that separates the saline solution into which the patient's eye is immersed from the distilled water in the imaging machine. The speculum comprises opposing pads 704a and 704b optionally comprising an adhesive to adhere to skin of a patient and a flexible eyelid retraction member 708a engaging the opposing pads 704a and 704b.

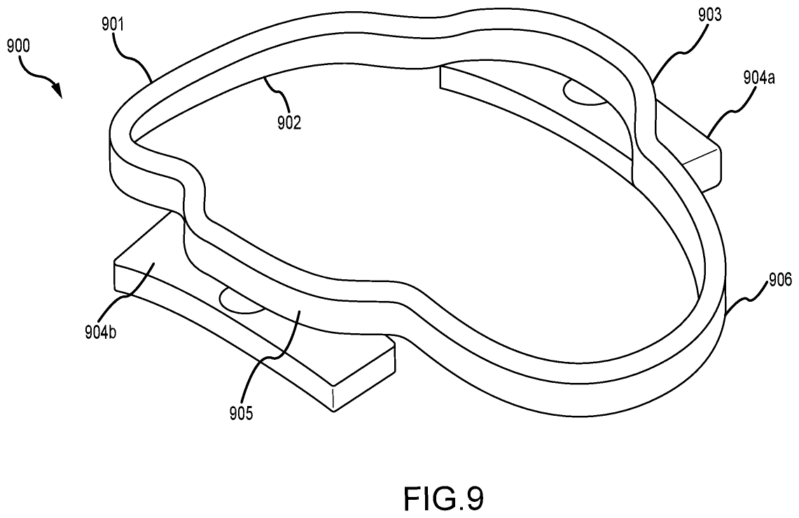

FIG. 9 is a schematic of the speculum of the embodiment. The long dimension of this speculum is about 1.70 inches. The speculum shown is typically fabricated from a thermoplastic plastic by a process such as injection molding. The speculum comprises opposing pads 904a and 904b optionally comprising an adhesive to adhere to skin of a patient and a flexible eyelid retraction member 908 engaging the opposing pads 904a and 904b. The retraction member 908 comprises a plurality of curved surfaces 901, 903, 905 and 906 collectively sized to surround an eye of the patient and press outwardly against the eyelids of the patient to urge the upper and lower eyelids to retract from the eye.

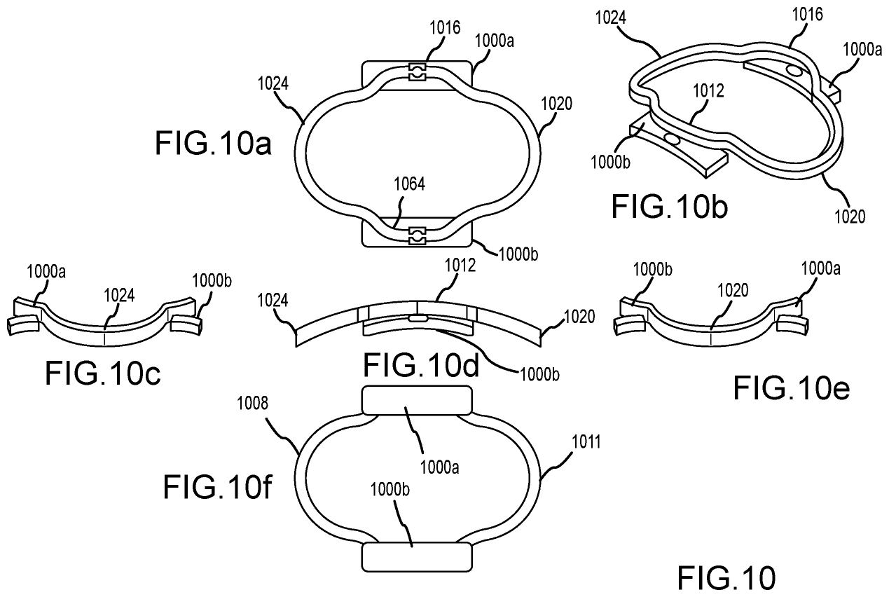

FIG. 10 shows various schematic views of the speculum of the embodiment. FIG. 10a is a top view of the speculum and FIG. 10f is a bottom view of the speculum. FIG. 10d is a side view of the speculum. As shown, the long dimension of the speculum is about 1.70 inches. FIG. 10b is an isometric view of the speculum and FIGS. 10c and 10e are end views of the speculum. As shown, the width dimension of the speculum is about 1.05 inches and the height dimension of the speculum is about 0.7 inches.

FIG. 10a is a top view of the speculum 1000 showing the opposing pads 1000a and 1000b. FIG. 10a also illustrates the shamrock shape of the flexible eyelid retraction member 1008 (see FIG. 9), and FIG. 10d is a side view of the speculum 1000 showing the arcuate, or vaulted, shape of the speculum 1000 with the bottom surface 1050 of the speculum engaging the patient's eyelids and skin around the eye and the upper surface 1054 of the speculum engaging the eyepiece. As shown, the long dimension of the speculum in FIG. 10d is about 1.70 inches. FIG. 10b is an isometric view of the upper surface of the speculum 1000 and FIGS. 10c and 10e are end views of the speculum 1000 taken from either opposing end of the speculum in FIG. 10d. As shown, the width dimension of the speculum (or the distance between the opposing pads 1000a,b) is about 1.05 inches and the height dimension of the speculum 1000 is about 0.7 inches taken from the lowest to the highest points 1060 and 1064, respectively, of the speculum of FIG. 10d. As can be seen from FIG. 10, the speculum comprises opposing pads that engage the patient's eyelids and interior surfaces of the eyepiece to maintain the speculum in a desired position for scanning.

A radius of curvature of each of opposing first and second curved surfaces 1020 and 1024 of the retraction member are typically substantially equivalent but each is greater than a radius of curvature of each of opposing third and fourth curved surfaces 1012 and 1016 of the retraction member. The radii of curvature of each of the third and fourth curved surfaces 1012 and 1016 are substantially equivalent. A first of the opposing pads 1004b engages the third curved surface 1012 and a second of the opposing pads 1004a engages the fourth curved surface 1016.

As shown in FIG. 10d, upper and lower surfaces 1054 and 1050 of the flexible eyelid retraction member 1008 and the opposing pads 1004a,b curve upwardly above a line defined by lower extremities 1060 of the first and second curved surfaces and an apex 1064 of the upward curve is located on the opposing third and fourth curved surfaces. The upward curve, when the flexible eyelid retraction member is in position on the patient, curves outwardly to substantially contact skin surrounding an eye socket of the patient. A radius of curvature of the upward curve is greater than the radius of curvature of each of the first, second, third and fourth curved surfaces.

FIG. 10f depicts a speculum according to another embodiment. The speculum is vaulted as in FIG. 10d but comprises only first and second curved or arcuate surfaces, each having ends coupled to the opposing pads. FIG. 8 depicts an eye seal in which the speculum is positioned in the absence of the patient to depict the relative position of the speculum with interior surfaces of the eye seal. In the speculum 700 of FIGS. 7-8 and 10f, the retraction member comprises first and second arcuate surfaces 708a, b, first and second ends of each connecting to the opposing pads 704a and 704b.

In the speculums of both FIGS. 10a-e and FIGS. 7, 8, and 10d, the flexible eyelid retraction member is sized to surround the eye of the patient and press outwardly against the eyelids of the patient and urge the upper and lower eyelids to retract from the eye. The first and second opposing pads engage skin adjacent to inner and outer ends of the upper and lower eyelids while the retraction member contacts the upper and lower eyelids above and below the iris, respectively. As noted, the flexible eyelid retraction member is configured to curve outwardly while in contact with the patient to substantially continuously contact skin surrounding the eye of the patient.

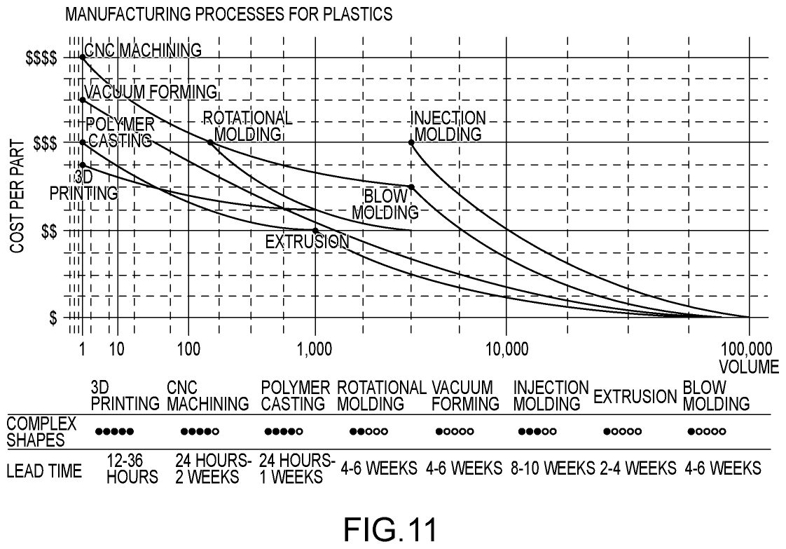

FIG. 11 illustrates approximate cost versus volume for several plastic fabrication processes. Current 3D printing technology is good for low volume prototyping whereas injection molding is appropriate for low cost, high volume production.

A thermoplastic, or thermo-softening plastic, is a plastic polymer material that becomes pliable or moldable at a certain elevated temperature and solidifies upon cooling.

Thermoplastics include:
Acrylic, also known by trade names such as Lucite, Perspex and Plexiglas.
ABS or acrylonitrile butadiene styrene
Nylon
Polylactic acid (polylactide) is one of the materials used for 3D printing.
Polybenzimidazole Polybenzimidazole is a synthetic fiber with a very high melting point.
Polycarbonate known under trademarks such as Lexan, Makrolon, Makroclear, and arcoPlus.
Polyether sulfone (PES) or polysulfone
Polyoxymethylene Polyoxymethylene (POM).
Polyetherether ketone (PEEK)
Polyetherimide Polyetherimide (PEI),
Polyethylene as ultra-high-molecular-weight polyethylene (UHMWPE); high-density polyethylene (HDPE); medium-density polyethylene (MDPE); low-density polyethylene (LDPE); and linear low-density polyethylene (LLDPE)
Polyphenylene (PPO)
Polyphenylene sulfide (PPS)
Polypropylene (PP)
Polystyrene (PS)
Polyvinyl chloride (PVC) and chlorinated polyvinyl chloride (CPVC)
Polyvinylidene fluoride, PVDF
Teflon (PTFE)

Manufacturing methods include:
3D printing which can include any number of specific processes such as SLA
(stereolithograpy), FDM (fused deposition modeling), MSLA (liquid crystal mask) and DLP
(digital light processing)
polymer casting
vacuum forming.
rotational molding
extrusion
injection molding

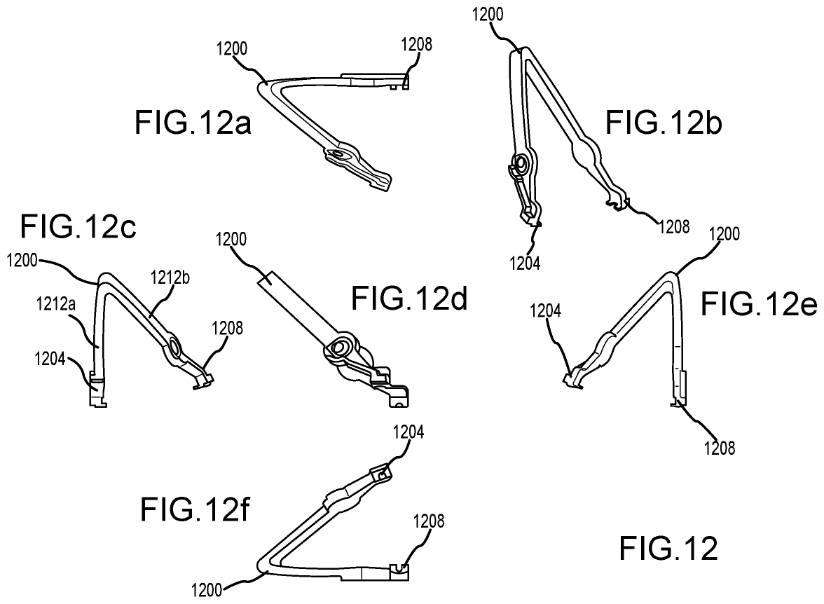

FIG. 12 shows various schematic views of a typical speculum applicator tool 1200 used to install the speculum of the embodiment into an eye piece. As shown in FIG. 12, each of the opposing ends 1204 and 1208 of the opposing arms 1212a and 1212b of the applicator tool are bent inwardly and slotted to form opposing pairs of clamping hooks that hold the opposing pads 1000a and 1000b (see FIG. 10) of the speculum securely for proper placement around the patient's eye. The opposing arms 1212a and 1212b of the applicator tool are articulated to allow the installer of the speculum more flexibility when installing the speculum in the eye piece. The opposing arms 1212a and 1212b of the applicator tool 1200 move towards and away from one another and function like a pair of tweezers to grasp the speculum to install the speculum into an eye piece (see FIGS. 7 and 8 showing the speculum as it would be installed in an eye piece).

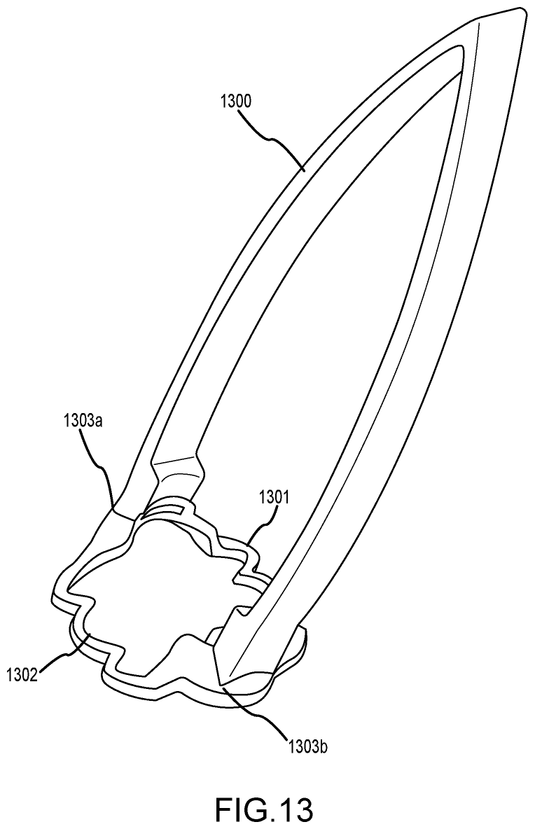

FIG. 13 is a schematic of a speculum applicator tool 1300 clamping on a version of the speculum of the embodiment 1301. As will be appreciated, the flexible speculum elastically deforms under inward pressure applied by the opposing arms 1303a and 1303b such that the inwardly facing corners of the speculum move towards one another as shown in FIG. 13. When the speculum is applied at the proper position on the patient's eye and the pressure released, the energy stored in elastic deformation of the speculum is released (like a spring) to enable the speculum to return to its original shape such as shown in FIG. 10a. This beneficially draws the eyelids and skin surrounding the eye back to expose the eye for scanning in the eyepiece.

The steps from applying an eye lid strip to engaging the eye seal with speculum are:

1. Attach the speculum to the applicator (see FIG. 13).
2. Remove the protective tabs from the tape on the top and bottom lid pads of the speculum.
3. Instruct the patient to close their eye and look down.
4. Squeeze the applicator to close the speculum (see FIG. 13).
5. Tilt the speculum and apply the top pad of the speculum to the upper eye lid as close to the edge of the eye lid as possible.
6. Instruct the patient to look up.
7. Push the upper eye lid upward as needed and apply the bottom pad to the lower eye lid as close to the edge of the eye lid as possible.
8. Release the speculum from the applicator.

The patient can now engage with the eye seal and scanning can proceed.

One of the approaches to applying the speculum of the present disclosure on a patient is to slightly compress the speculum so as to apply it to the patient's eyelid and to do so without bringing the hand of the operating technician or doctor too close to the patient's eye. One means to accomplish this to form a plastic applicator tool in the same process used to fabricate the speculum. For example, an injection mold could be used to mold an applicator tool and a speculum simultaneously. The plastic tool would act as a pair of tweezers to grasp and slightly compress the speculum for application while keeping the hand of the operating technician or doctor from touching the patient's eye. This would facilitate putting the speculum under the patient's eyelid prior to the patient placing his or her eye against an eyepiece attached to a precision ultrasound imaging device as shown for example in FIGS. 5 and 6.

A number of variations and modifications of the disclosure can be used. As will be appreciated, it would be possible to provide for some features of the disclosures without providing others.

The present disclosure, in various embodiments, includes components, methods, processes, systems and/or apparatus substantially as depicted and described herein, including various embodiments, sub-combinations, and subsets thereof. Those of skill in the art will understand how to make and use the present disclosure after understanding the present disclosure. The present disclosure, in various embodiments, includes providing devices and processes in the absence of items not depicted and/or described herein or in various embodiments hereof, including in the absence of such items as may have been used in previous devices or processes, for example for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover though the description of the disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. A speculum, comprising:
opposing pads comprising an adhesive to adhere to skin of a patient; and
a flexible eyelid retraction member engaging the opposing pads, the retraction member comprising a plurality of curved surfaces collectively sized to surround an eye of the patient and press outwardly against the eyelids of the patient to urge the upper and lower eyelids to retract from the eye.

2. The speculum of claim 1, wherein a radius of curvature of each of opposing first and second curved surfaces of the retraction member is greater than a radius of curvature of each of opposing third and fourth curved surfaces of the retraction member.

3. The speculum of claim 2, wherein a first of the opposing pads engages the third curved surface and a second of the opposing pads engages the fourth curved surface.

4. The speculum of claim 2, wherein upper and lower surfaces of the flexible eyelid retraction member and the opposing pads curve upwardly above a line defined by lower extremities of the first and second curved surfaces and an apex of the upward curve is located on the opposing third and fourth curved surfaces.

5. The speculum of claim 4, wherein a radius of curvature of the upward curve is greater than the radius of curvature of each of the first, second, third and fourth curved surfaces.

6. The speculum of claim 1, wherein the speculum is received by an eye seal of an ocular imaging device.

7. The speculum of claim 1, wherein the retraction member comprises first and second arcuate surfaces, first and second ends of each connecting to the opposing pads.

8. A speculum, comprising:
opposing pads contacting first portions of the eyelids of a patient; and
a flexible eyelid retraction member engaging the opposing pads and contacting second portions of the patient's upper and lower eyelids, the first and second portions being discrete from each other, wherein the flexible eyelid retraction member curves outwardly to substantially contact skin surrounding an eye socket of the patient.

9. The speculum of claim 8, wherein the retraction member comprises a plurality of curved surfaces collectively sized to surround an eye of the patient and press outwardly against the eyelids of the patient to urge the upper and lower eyelids to retract from the eye.

10. The speculum of claim 9, wherein a radius of curvature of each of opposing first and second curved surfaces of the retraction member is greater than a radius of curvature of each of opposing third and fourth curved surfaces of the retraction member.

11. The speculum of claim 10, wherein a first of the opposing pads engages the third curved surface and a second of the opposing pads engages the fourth curved surface.

12. The speculum of claim 10, wherein upper and lower surfaces of the flexible eyelid retraction member and the opposing pads curve upwardly above a line defined by lower extremities of the first and second curved surfaces and an apex of the upward curve is located on the opposing third and fourth curved surfaces.

13. The speculum of claim 12, wherein a radius of curvature of the upward curve is greater than the radius of curvature of each of the first, second, third and fourth curved surfaces.

14. The speculum of claim 8, wherein the speculum is received by an eye seal of an ocular imaging device.

15. The speculum of claim 9, wherein the retraction member comprises first and second arcuate surfaces, first and second ends of each connecting to the opposing pads.

16. A speculum, comprising:
first and second opposing pads; and
a flexible eyelid retraction member engaging the first and second opposing pads, the retraction member comprising a plurality of curved surfaces collectively sized to surround an eye of the patient and press outwardly against the eyelids of the patient and urge the upper and lower eyelids to retract from the eye, wherein the first and second opposing pads engage skin adjacent to inner and outer ends of the upper and lower eyelids while the retraction member contacts the upper and lower eyelids above and below the iris, respectively, and wherein the flexible eyelid retraction member is configured to curve outwardly while in contact with the patient to substantially continuously contact skin surrounding the eye of the patient.

17. The speculum of claim 16, wherein a radius of curvature of each of opposing first and second curved surfaces of the retraction member is greater than a radius of curvature of each of opposing third and fourth curved surfaces of the retraction member.

18. The speculum of claim 17, wherein the first opposing pad engages the third curved surface and the second opposing pad engages the fourth curved surface.

19. The speculum of claim 17, wherein upper and lower surfaces of the flexible eyelid retraction member and the opposing pads curve upwardly above a line defined by lower extremities of the first and second curved surfaces and an apex of the upward curve is located on the opposing third and fourth curved surfaces.

20. The speculum of claim 19, wherein a radius of curvature of the upward curve is greater than the radius of curvature of each of the first, second, third and fourth curved surfaces.

21. The speculum of claim 16, wherein the speculum is received by an eye seal of an ocular imaging device.

22. The speculum of claim 16, wherein the retraction member comprises first and second arcuate surfaces, first and second ends of each connecting to the opposing pads.
